(19) United States
(12) Patent Application Publication
(10) Pub. No.: US 2009/0187164 A1
(43) Pub. Date: Jul. 23, 2009

(54) Nasogastric tube placement and monitoring system

(76) Inventor: Philip S. Rowe, Port St. Lucia, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 11/799,664

(22) Filed: May 2, 2007

Related U.S. Application Data
(60) Provisional application No. 60/797,307, filed on May 3, 2006.

(51) Int. Cl. A61M 25/098

(52) U.S. Cl. 604/529

(57) ABSTRACT

A nasogastric tube placement and monitoring system is provided including a flexible nasogastric tube and numerous circuits that can be used alone or in combination. The circuits include a feedback initiator, a feedback receiver, and a clinician notifying device. By various means in the provided circuits, the feedback initiator provides information about the location of the distal end of the nasogastric tube. This information or data is received and analyzed by the feedback receiver that monitors the circuit, which then transmits an output to the clinician notifying device to alert or advise the attending clinician of this information. The data that is output supplies information about the location of the tube's distal end to the clinician, thereby assisting the clinician in placement of the nasogastric tube during intubation, as well as in monitoring proper tube placement after placement.

### CROSS-REFERENCE TO RELATED APPLICATION

This application claims the benefit of co-pending U.S. Provisional Patent Application Ser. No. 60/797,307, filed on May 3, 2006, which is incorporated herein in its entirety.

### BACKGROUND OF THE INVENTION

1. Field of the Invention

The present invention relates generally to a medical device, a nasogastric tube for use in intubation of patients, and more particularly, to a nasogastric tube placement and monitoring system configured to assure proper placement—either in the stomach or in the lungs—and monitoring of a nasogastric tube (NG-tube) through pH/continuity, auscultatory feedback, air pressure sensors, contact pressure sensors, and indicator lights, meters, and/or speakers. A nasogastric tube is a plastic tubular conduit inserted through the nose, down through the alimentary canal into the stomach. Nasogastric tubes can also be inserted directly into the lung(s) for therapeutic purposes. Therefore the organ of interest is either the stomach or lungs when considering the use of a nasogastric tube. When the organ of interest is the stomach, the nasogastric tube is used to either deliver hydration, nutrition, and medications to patients or to suction toxic material from the stomach. Nasogastric tubes are also used therapeutically to suction material from the lungs.

2. Description of the Prior Art

The use of a nasogastric tube, a plastic tubular conduit inserted through the nose into the throat down through the alimentary canal and into the stomach, is an important standard therapeutic technique. The nasogastric tube is commonly used to introduce materials such as nutrition, hydration, or medications into the stomach. It is also used to decompress the stomach to prevent vomiting after major surgery and for removing material from the body, such as an accidentally ingested poison, an overdose of drugs, or other toxins that have built up in the stomach, perhaps due to surgery, or gastrointestinal blockages. It is widely recognized that enteral nutrition provided by a nasogastric tube, or a feeding tube as it is sometimes referred to, is preferred to parenteral nutrition, as nasogastric tubes present less complications. Nasogastric tubes are also used for other appropriate therapeutic actions where the lungs may be the organ of interest. For example, the nasogastric tube may be inserted into the lungs for the purpose of suctioning or draining fluid if a patient has pneumonia or for the purpose of suctioning out toxins.

Although nasogastric intubation is a widely used, standard technique in hospitals, long term care facilities, hospices, and in-home health care delivery, it continues to be challenging for clinicians to assure proper placement of a nasogastric tube. Great care and attention must be used to assure the tube is correctly placed.

Inadvertent placement of a nasogastric tube has caused numerous health problems for patients. These include inadvertent placement of the nasgastric tube into the tracheobronchial tree or pleural space, when the organ of interest for placement was the stomach. Aspiration by proxy is a serious consequence of inadvertent misplacement. This occurs when food or medicine is introduced by a tube that is incorrectly placed into the lungs, trachea, or esophagus, leading to dangerous aspirated pneumonia with its associated increased incidence of morbidity and mortality. Improper tube placement has led to laryngeal injuries due to placement in the trachea and distal airways, when the organ of interest was the stomach. Other related issues include hypersalivation, depressed cough reflex, and pharyngeal abnormalities.

Determining correct placement is vital. Complications from improper tube positioning often result in extended hospital stays, or in some instances, results in death.

The importance of proper intubation procedures and the difficulty of assuring correct placement have led to the development of numerous confirmation techniques that are used either individually or in combination with others to assess proper nasogastric tube placement. Some of these include capnometry, capnography, auscultation, and endoscopic fluoroscopic techniques.

Radiographic confirmation of the location of the distal end of the tube, however, is the most reliable confirmation technique. Even when an experienced clinician blindly places a nasogastric tube, placement must then be verified by radiographic confirmation. A standard tube has a radio-opaque marker or strip at the distal end, so the position can be verified by X-ray studies of the chest/abdomen. If the X-ray cannot confirm the position, an alternative technique known as fluoroscopy can be used to confirm the distal end location. Radiographic assistance can also be used during the insertion of the tube.

While radiographic confirmation does assure correct placement of a nasogastric tube, the patient is exposed to radiation, and the cost of radiographic confirmation is costly and radiographic confirmation is difficult or impossible in some situations, such as, for example, some in-home health care. Additionally, some patients that require nasogastric tubes have multiple pieces of life support equipment. Therefore, a substantial amount of time, effort, and hospital staff are required to move, position, and manage these patients while performing the radiographic confirmation.

It would be advantageous to provide a method for safe and correct placement and monitoring of a nasogastric tube into the organ of interest, either the stomach or lung, while incurring a significantly lower cost than the traditional radiographic confirmation. This would also reduce or eliminate the need for radiographic confirmation. The need for specialized staff to perform the various placement and monitoring techniques could also be greatly reduced. A method that can provide continuous verification of the position of the distal end of the nasogastric tube, both during intubation, and during the entire intubation period would be advantageous for both the patient and the attending hospital staff.

A traditionally used bedside technique to evaluate the placement of a nasogastric tube placement is auscultation of air insufflated through the tube. In this method a trained technician using a stethoscope above the stomach, rapidly fills the tube with a bolus of air, and determines whether the sound generated by the air injected into the tube is from the gastrointestinal system, from the respiratory system, or other location. This is a very economical test method, but the amount of training and clinical experience required is substantial. Additionally, this method is very time consuming, as the trained clinician attempts to correctly differentiate the sounds to determine the location of the distal end of the nasogastric tube. Furthermore, this method does not deliver a high degree of accuracy.

Another placement evaluation method involves aspiration of fluid from the tube, with pH testing of the aspirate. By using pH paper the acidity of the fluid can be determined. An acidic pH of approximately lower than 5 indicates the correct placement into the stomach, while an aspirate of pH 6 or greater indicates a tube inadvertently positioned in the respiratory system. One problem associated with this method of using the aspirate of the tube is the tendency for small-bore tubes to collapse when suction is applied. Additionally, aspirating fluid requires a significant investment of time and effort by the trained clinician. Also, it is difficult to obtain an aspirate from the tube in dehydrated patients or in certain areas of the stomach where there may be no pool of fluid of sufficient volume to aspirate. It would be advantageous to have a device that decreased the amount of time spent by hospital personnel to aspirate fluid and to test the pH of the fluid every time a pH value was desired.

Even after a successful initial placement of a nasogastric feeding tube is confirmed, the patient faces an ongoing risk. This is because over time the distal end of the tube can become mal-positioned, moving from its original location. For example, this may occur due to patient movement or the patient may dislodge the tube because it is uncomfortable. Commonly hospital policies recommend frequent and ongoing placement confirmation, for example before every feeding or at least every six hours. Obviously a great deal of radiation exposure would be received if this confirmation were done by radiology, as well as being financially costly. It would be advantageous to have a device for continual monitoring of the location without the expense and the radiation exposure of repeated X-rays.

Accordingly, there is an established need for a timesaving, economical, nasogastric tube placement and monitoring system that will guide a clinician during the placement of the nasogastric tube, so that proper placement is achieved into the organ of interest, whether it is the stomach or the lungs, and, additionally, to provide proper monitoring during the entire intubation period, while minimizing radiological confirmation as the main procedure to assure proper placement.

### SUMMARY OF THE INVENTION

The present invention is directed to an economical, time-saving, efficient, nasogastric tube placement and monitoring system that is capable of assisting clinicians with proper tube placement during intubation and also of advising the clinician during the entire intubation period, by continually monitoring and providing information regarding the location of the distal end of the tube. This system provides a flexible nasogastric tube and numerous circuits that can be used alone or in combination. The circuits include a feedback initiator, a feedback receiver, and a clinician notifying device. By various means in the provided circuits, the feedback initiator provides information about the location of the distal end of the nasogastric tube. This information or data is received by the feedback receiver that monitors the circuit. The feedback receiver receives the information about the location of the distal end of the nasogastric tube from the feedback initiator, and transmits an output to a clinician notifying device to alert or advise the attending clinician of this information. In most provided circuits a conducting means is incorporated into, or connected to, the nasogastric tube to connect the feedback initiator to the feedback receiver.

In the provided systems the feedback initiator may be any of a variety of devices, such as, for example, a pH sensor, an air pressure sensor, a contact pressure sensor, a continuity circuit, a crystal for feedback for monitoring conductor integrity, a transducer to produce sound waves or tones, or a vibration device that provides a sound which can be heard through a stethoscope. The feedback receiver may be any of a variety of devices, such as, for example, a pH monitor, a continuity monitor, a contact pressure monitor, an air pressure monitor, a stethoscope diaphragm, or a handheld tone receiver. The output clinician notifying device may also be any of a variety of devices, such as, for example, a digital readout display, a speaker, warning lights, indicating lights, or an alarm. The data that is output by the notifying device supplies information about the location of the tube's distal end to the clinician and thereby assists the clinician in placement of the nasogastric tube, as well as in monitoring after placement.

An object of the present invention is to provide a nasogastric tube placement and monitoring system that confirms nasogastric tube placement without the use of radiological confirmation.

An additional object of the present invention is to provide a nasogastric tube placement and monitoring system that minimizes the patient's exposure to radiation.

A further object of the present invention is to provide a nasogastric tube placement and monitoring system that provides a significantly lower cost than the cost of the traditional radiographic confirmation.

An additional object of the present invention is to provide a nasogastric tube placement and monitoring system that minimizes patient health risks associated with improper placement.

Another object of the present invention is to provide a nasogastric tube placement and monitoring system that is configured to continuously monitor the location of the distal end of the tube.

A further object of the present invention is to provide a nasogastric tube placement and monitoring system that increases staff efficiency.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a perspective view showing an overview of the preferred embodiments of the nasogastric tube placement and monitoring system of the present invention in a typical application illustrating placement in the stomach;

FIG. 2 is a perspective diagrammatic view showing the pH monitoring circuit/continuity circuit of the first preferred embodiment of the nasogastric tube placement and monitoring system of the present invention as implemented with a distal pH sensor and/or continuity conductor;

FIG. 3 is a diagrammatic flowchart showing the pH monitoring circuit/continuity circuit of the first preferred embodiment of the nasogastric tube placement and monitoring system of the present invention;

FIG. 4 is a perspective diagrammatic view showing the audio monitoring circuit of the second preferred embodiment of the nasogastric tube placement and monitoring system of the present invention illustrating an audio monitoring circuit as implemented with a proximal acoustic feedback receiver, such as a microphone;

FIG. 5 is a diagrammatic flowchart showing the audio monitoring circuit of the second preferred embodiment of the nasogastric tube placement and monitoring system of the present invention;

FIG. 6 is a perspective view showing the pressure sensor circuit of the fourth preferred embodiment of the nasogastric tube placement and monitoring system of the present invention as implemented with a distal contact pressure sensor;

FIG. 7 is a diagrammatic flowchart showing the pressure sensor circuit of the fourth preferred embodiment of the nasogastric tube placement and monitoring system of the present invention;

FIG. 8 is a perspective diagrammatic view showing air pressure sensor circuit of the fifth preferred embodiment of the nasogastric tube placement and monitoring system of the present invention illustrating an air pressure circuit as implemented with a proximal air pressure sensor;

FIG. 9 is a diagrammatic flowchart showing the air pressure sensor circuit of the fifth preferred embodiment of the nasogastric tube placement and monitoring system of the present invention;

FIG. 10 is a perspective diagrammatic view showing a variation in characteristics of the preferred flexible tube to be used with any of the preferred embodiments of the nasogastric tube placement and monitoring system of the present invention illustrating a crystal and a heat-sensitive color indicator;

FIG. 11 is a front diagrammatic view showing the control panel of the nasogastric tube placement and monitoring system of the present invention, illustrating the use of a combination of the circuits of the present invention, as implemented with a combination of feedback initiators, feedback receivers, and notifying devices;

FIG. 12 is a diagrammatic flowchart showing the stomach monitoring circuit of the seventh preferred embodiment of the nasogastric tube placement and monitoring system of the present invention;

FIG. 13 is a diagrammatic flowchart showing the lung monitoring circuit of the eighth preferred embodiment of the nasogastric tube placement and monitoring system of the present invention;

FIG. 14 is a diagrammatic flowchart showing the location monitoring circuit of the ninth preferred embodiment of the nasogastric tube placement and monitoring system of the present invention;

FIG. 15 is a perspective diagrammatic view of the third embodiment of the nasogastric tube placement and monitoring system of the present invention, illustrating the vibration circuit;

FIG. 16 is a diagrammatic flow chart illustrating the vibration circuit of the third preferred embodiment of the nasogastric tube placement and monitoring system of the present invention;

FIG. 17 is a perspective diagrammatic view showing the sixth preferred embodiment of the nasogastric tube placement and monitoring system of the present invention, illustrating a wave generator and receiver circuit;

FIG. 18 is a diagrammatic flow chart showing the wave generator and receiver circuit of the sixth preferred embodiment of the nasogastric tube placement and monitoring system of the present invention; and

FIG. 19 is a chart showing elements of the provided circuits of the nasogastric tube placement and monitoring system of the present invention.

Like reference numerals refer to like parts throughout the several views of the drawings.

### DETAILED DESCRIPTION OF THE PREFERRED EMBODIMENTS

Shown throughout the figures, the present invention is directed toward a nasogastric tube placement and monitoring system that provides information about the location of the distal end of the nasogastric tube. The nasogastric tube placement and monitoring system of the present invention is not only capable of assisting clinicians during intubation, but also capable of allowing clinicians to determine the placement after intubation, and, additionally, continually during the hours or days that a nasogastric (NG) tube is being utilized. A variety of systems or circuits are provided, which may be used individually or in any combination to provide the clinician with optimum information concerning the location of the distal end of the nasogastric tube.

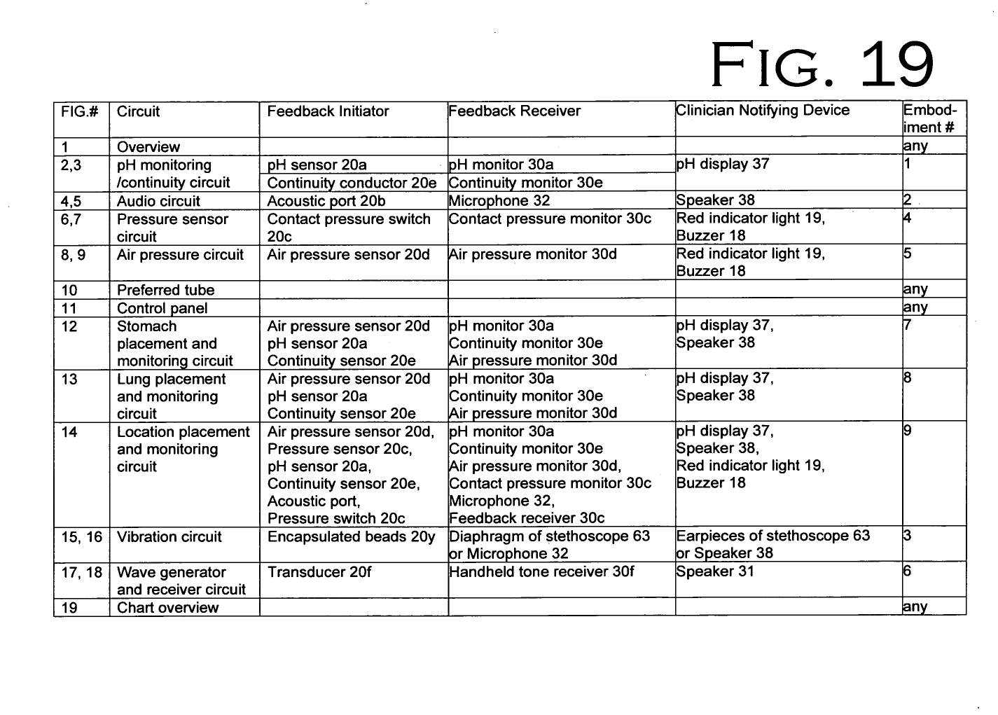

For clarity, a chart giving a generalized summary and overview of the various circuits and their elements is provided in FIG. 19. While the various systems or circuits provided are described or identified by the term “circuit”, which may refer to a path of an electric current, the term is herein used more broadly to also encompass a path of other data or information, such as, for example, sound waves.

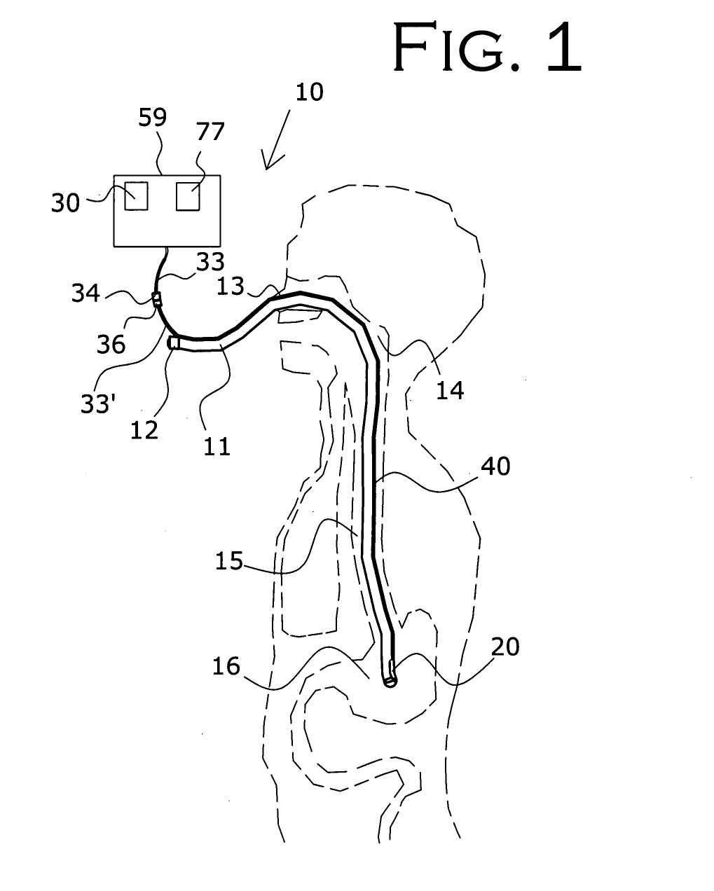

Referring now to FIG. 1, a nasogastric tube placement and monitoring system, shown generally as reference number 10, is illustrated as an overview of the preferred embodiments of the present invention. As shown, the nasogastric tube placement and monitoring system 10 of the present invention generally includes a flexible tube 11, a feedback initiator 20, a feedback receiver 30, a conducting means 40, a clinician notifying device 77, and, preferably, a user interface, control panel 59.

The standard nasogastric flexible tube as is known in the art, as well as the nasogastric flexible tube 11 of the present invention, has a distal end which is inserted into the patient, a proximal end which remains outside the patient, and an extended midsection to provide sufficient length for the intubation procedure. Both the standard nasogastric tube and the nasogastric flexible tube 11 of the present invention are adapted and sized for insertion into a nasal passage of a patient. The flexible tube 11 can be of any conventionally available sizes or materials, and includes single, double, and triple lumen tubes, as are known in the art. If desired for the intended procedure, the proximal end of flexible tube 11 may be configured with a standard adapter 12 for connection to standard medical equipment or supplies, for example ready-to-hang, enteral nutrition containers.

The nasogastric flexible tube 11 of the present invention is further configured with the feedback initiator 20, which is distally located in most provided circuits.

In most circuits provided, the flexible tube 11 is configured with the conducting means 40 running along the length of flexible tube 11, incorporated into, or connected to flexible tube 11. The conducting means 40 extends past the distal end of flexible tube 11 in an extended portion 33, 33′.

Preferably, between the extended portion 33 of conducting means 40 and the extended portion 33′ of conducting means 40, complementary fittings of connectors 36, 34 are provided, which are preferably quick connectors for the convenience of the assisting clinician. The proximal end of conducting means 40 preferably terminates at control panel 59, while the distal end preferably terminates at the distal end of tube 11, which, in most provided circuits, is near feedback initiator 20. Generally conducting means 40 extends between and electrically connects feedback initiator 20 to feedback receiver 30 via connectors 36, 34, with a portion of conducting means disposed along tube 11 and an extended portion 33, 33′ extending on to the feedback receiver 30. Conducting means 40 is appropriately configured for proper operation, and preferably is appropriately shielded.

In the nasogastric tube placement and monitoring system of the present invention the control panel 59 is configured to be placed near a patient's bed at an easily readable and accessible location. The control panel 59 serves as a user interface, allowing the clinician to easily and conveniently manually initiate the one or more circuits to be used in the nasogastric tube placement and monitoring system of the present invention, as well as providing the feedback from the distal end of the nasogastric tube to the clinician in an accessible and readily available manner.

Control panel 59 integrates the various elements of the one or more (preferably multiple) circuits used for the nasogastric tube placement and monitoring system, providing a housing for the required monitoring electronics and for the clinician notifying devices such as displays or speakers, as utilized by the circuit or circuits being utilized. Control panel 59 is configured with the necessary electronics for the circuits and systems provided in the nasogastric tube placement and monitoring system of the present invention. Preferably, control panel 59 is incorporated into an aesthetically pleasing cabinet or enclosure that is easy to clean, to maintain, and to move to the location of use.

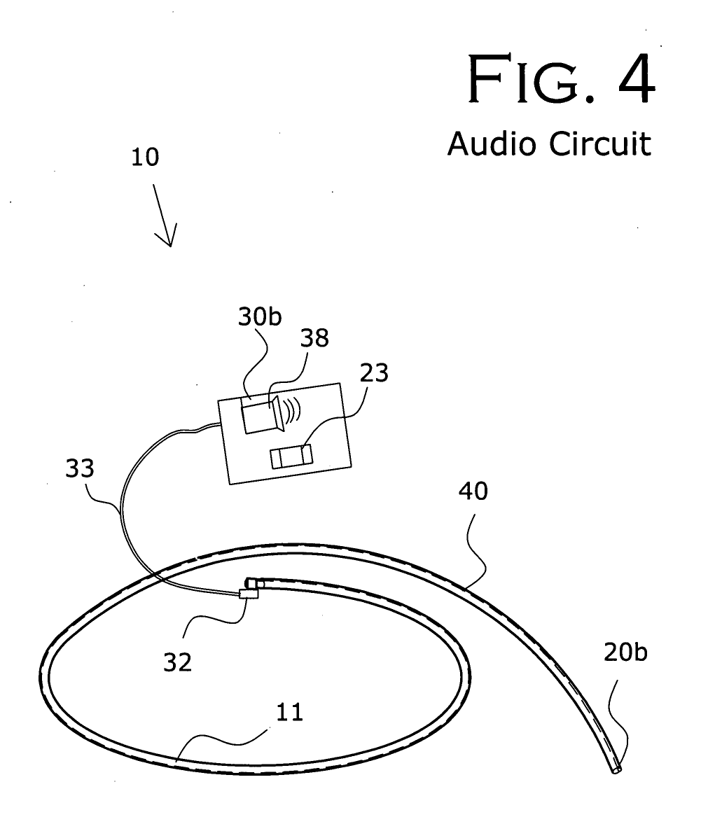

Feedback receiver 30 is preferably disposed in control panel 59. Feedback receiver 30, in most provided circuits, is electrically connected to feedback initiator 20 through connectors 34, 36 via conducting means 40. Connectors 34, 36 serve to allow feedback receiver 30, with its associated wiring of extended portion 33, to be easily engaged and disengaged from the proximal end of flexible tube 11. Connectors 34, 36 allow flexible tube 11 to be disposable, while feedback receiver 30 may be reusable or, optionally, disposable. Conducting means 40 and wiring extended portion 33, 33′ are configured with the appropriate wiring and connecting fittings to transport signals between feedback initiator 20 and feedback receiver 30 via connectors 34, 36. Optionally, conducting means 40 can be directly electrically connected to feedback receiver 30 without the utilization of connectors 34, 36 as in FIG. 4, although connectors 34, 36 are preferred for convenience of tube 11 replacement.

The clinician uses the standard general method of placing a nasogastric tube into the proper location, which is the organ of interest for the intended procedure. Most commonly, the organ of interest is the stomach, but at times, the organ of interest is the lung. For example, if the stomach is the organ of interest, the distal end of nasogastric tube 11 is inserted through the nose 13 into the throat 14 and then down the esophagus 15 into the stomach 16 of the patient. While the intubation procedure is being carried out, feedback receiver 30 receives information about the distal end of tube 11 from feedback initiator 20, and supplies that information to the clinician via the clinician notifying device 77, thereby assisting in and verifying the tube placement.

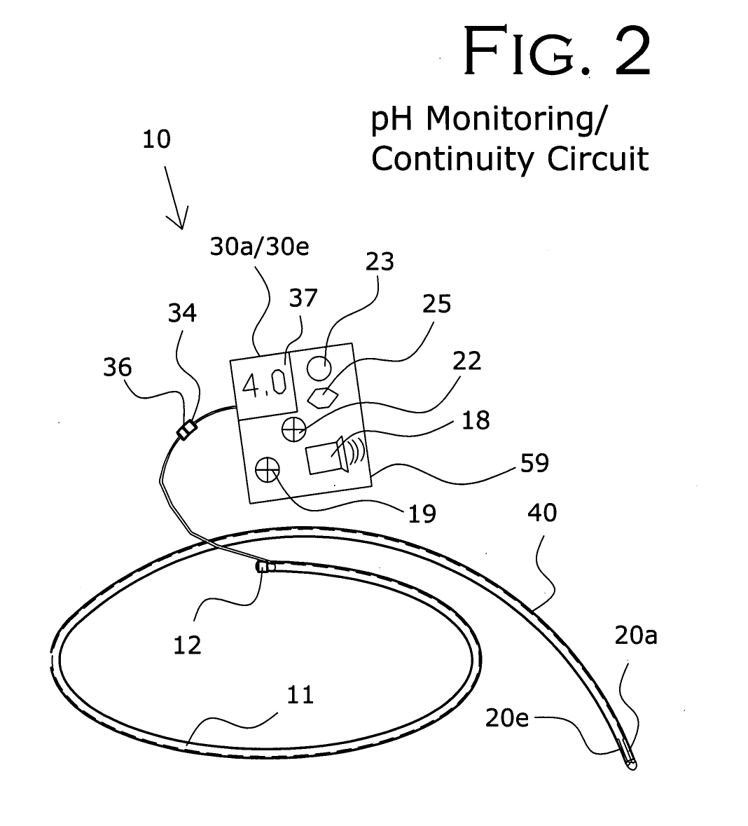

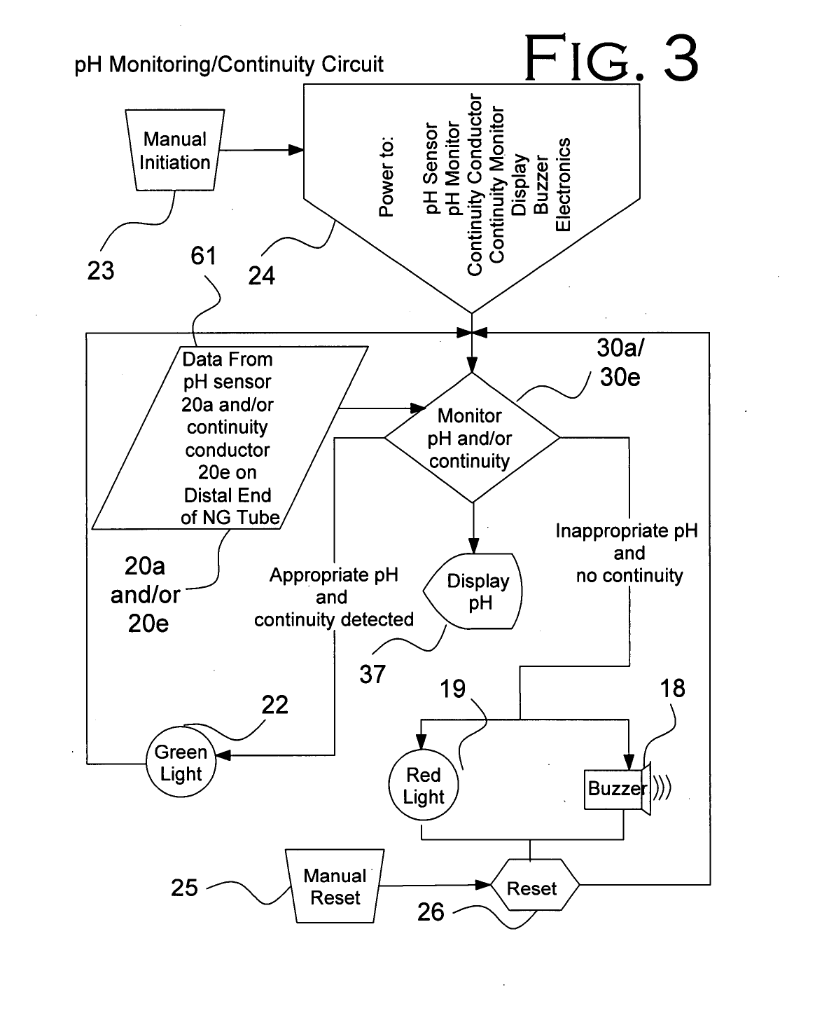

In the first embodiment, as seen in FIG. 2 and FIG. 3, a pH monitoring circuit and/or continuity circuit is provided. The feedback initiator 20 is a pH sensor 20a and/or continuity conductor 20e. PH sensor 20a continually senses the acidity or basicity of the fluid located at the distal end of flexible tube 11 by giving a measurement of the concentration of hydrogen ions. The continuity circuit, utilizing continuity conductor 20e, is a means of determining continuity through which the conductors continually monitor the conductivity of the fluid located at the distal end of the flexible tube 11, monitoring the presence of hydrogen ions.

The continuity circuit provides a potential to continuity conductor 20e at the distal end of flexible tube 11. Conducting means 40 is used as a means to provide or supply the potential to continuity conductor 20e, and, if continuity at continuity conductor 20e is available or detected, conducting means 40 further transmits the potential back to the proximal end of flexible tube 11 to continuity monitor 30e. Thus if conductivity is present during the presence of hydrogen ions, continuity monitor 30e is activated. The feedback receiver, continuity monitor 30e, configured with the appropriate electronics package, receives the electrical output from conducting means 40, analyzes it, and outputs a reading, displayed on pH display 37 or, alternately on a separate display (not shown), on control panel 59 that can be easily read by the clinician. The pH display 37 serves as the notifying device that provides the clinician with the information concerning the distal end of flexible tube 11.

Although shown as providing a digital readout on pH display 37, the pH display can be in the form of an analog meter. Furthermore, one or more indicator lights 19, 22 can be used to notify the clinician of the detected information. Additionally, the detected continuity can be utilized with other circuits in the nasogastric tube placement and monitoring system 10 of the present invention. The continuity circuit is configured with appropriate resistors, wiring, shielding for the wiring, and other electronics to determine whether continuity is detected at the distal end of flexible tube 11 via continuity conductor 20e.

Preferably an initiation button 23 is provided to manually start the power to the parts of the continuity circuit, as well as a reset button 25 to manually turn off the power.

The pH monitoring circuit uses a conventionally available pH sensor. Any pH sensor as is known in the art is within the scope of the invention, for example combination electrode sensors or solid-state pH electrode sensors. Preferably, pH sensor 20a is a combination pH sensor including a pH measuring electrode that is sensitive to the hydrogen ions and develops a potential or voltage directly related to the hydrogen ion concentration of the fluid, a reference electrode that provides a stable potential against which the measuring electrode can be compared, and a preamplifier that strengthens and stabilizes the signal. The two electrodes generate a voltage related to the pH of the fluid. After conditioning in the pre-amplifier, the electrical signal is transported by conducting means 40 along the length of flexible tube 11 and past the proximal end of flexible tube 11 to feedback receiver 30 via connectors 34, 36. A pH sensor preferably will be chosen that has a suitable resolution of approximately 0.1 pH, that has a suitable range of pH measurements, that is suitably protected in a housing, and that is designed for use in a fluid sample environment. Preferably, to save the time of the clinician at the time of intubation, the pH sensor 20a will not require calibration before use, although all pH sensors are within the scope of the invention including those requiring calibration.

A pH monitor 30a (the feedback receiver) configured with the appropriate electronics package, receives the electrical output from pH sensor 20a, analyzes it (particularly to determine if the detected pH is appropriate for the organ of interest), and outputs a display 37 of the detected pH value that can be easily read by the clinician. Although shown as providing a digital readout, feedback receiver 30a can be configured to provide either a digital readout or analog readout for display 37. Furthermore, one or more indicator lights 19, 22 can be used to notify the clinician of the detected pH information. Additionally, the sensed pH value can be utilized with other circuits in the nasogastric tube placement and monitoring system 10 of the present invention.

Optionally, the pH sensor 20a may be directly connected via conducting means 40 to the pH display 37, without the benefit of the pH monitor 30a, providing a simplified variation. In this case no analysis of the detected pH would be performed to determine if it was appropriate for the organ of interest, but the detected pH would be directly displayed to be read by the clinician.

Therefore the first embodiment of the nasogastric tube placement and monitoring system 10 provides the pH value at a specific point in time, as well as being a permanently installed sensor in a long term monitoring system. A lower pH reading, such as, for example, approximately pH 5 or lower, indicates the correct placement into the stomach, while a higher pH reading, such as, for example, approximately pH 6 or greater, indicates a tube 11 inadvertently positioned in the respiratory system. Thus the clinician can quickly determine the pH at the distal end of flexible tube 11 merely by looking at bedside display 37, instead of being required to aspirate fluid from the tube and to perform a test on it, as is currently the situation. Additionally, the pH measurement will be continually displayed while the tube 11 continues to be in place, allowing the clinician to refer to it any time desired without the time investment of aspirating fluid for testing and without the time and cost investment of radiological confirmation. Furthermore, the patient's exposure to radiation is minimized as this method reduces the need for X-rays.

To facilitate use of the pH monitoring, preferably an initiation button 23 is provided to commence the power to the parts of the pH monitoring circuit, as well as a reset button 25. Also, if the pH is inappropriate for the organ of interest, a warning or indicator light 19 and a buzzer 18 are provided. For example, if the pH is 7.5 when the placement location desired for the nasogastric tube is the stomach, the preferably red indicator light 19 and the buzzer 18 are activated by the pH monitoring circuit, thereby conveniently alerting the clinician. Optionally, either preferably red indicator light 19 or buzzer 18 may be utilized alone as indicators. Also optionally, a preferably green indicator light 22 may be provided to quickly allow the clinician to determine that the pH is appropriate to the organ of interest, without having to read the pH meter display 37. For convenience and efficiency, preferably the initiation button 23, reset button 25, indicator light 19, indicator light 22, buzzer 18, and pH meter display 37 are incorporated into control panel 59.

The flowchart of FIG. 3 illustrates an overview of the operation of the pH monitoring/continuity circuit. The pH monitoring/continuity circuit is started when the clinician engages a button or switch, manual initiation 23. This initiation provides power 24 to the pH sensor 20a, pH monitor 30a, continuity monitor 30e, continuity conductor 20e, pH display 37, buzzer 18 and the other electronic circuitry of the pH monitoring/continuity circuit. In block 30a/30e, the pH monitor 30a and continuity monitor 30e receive data from the pH sensor 20a and/or continuity conductor 20e (block 61) on the distal end of nasogastric tube 11, analyze the received detected pH based on the organ of interest, and determine if the detected pH is appropriate for the selected organ of interest and to activate the appropriate lights or buzzer.

In block 37, the received detected pH data is output and displayed on the pH meter display 37. If the pH and continuity is appropriate for the organ of interest, the indicator light 22 will be lighted (block 22). If the pH or continuity is inappropriate, the preferably red indicator light 19 will be lit and, preferably optional buzzer 18 will sound an audible alarm, which may be manually reset by the clinician by depressing reset button 25, whereby the electronics reset the circuit 26.

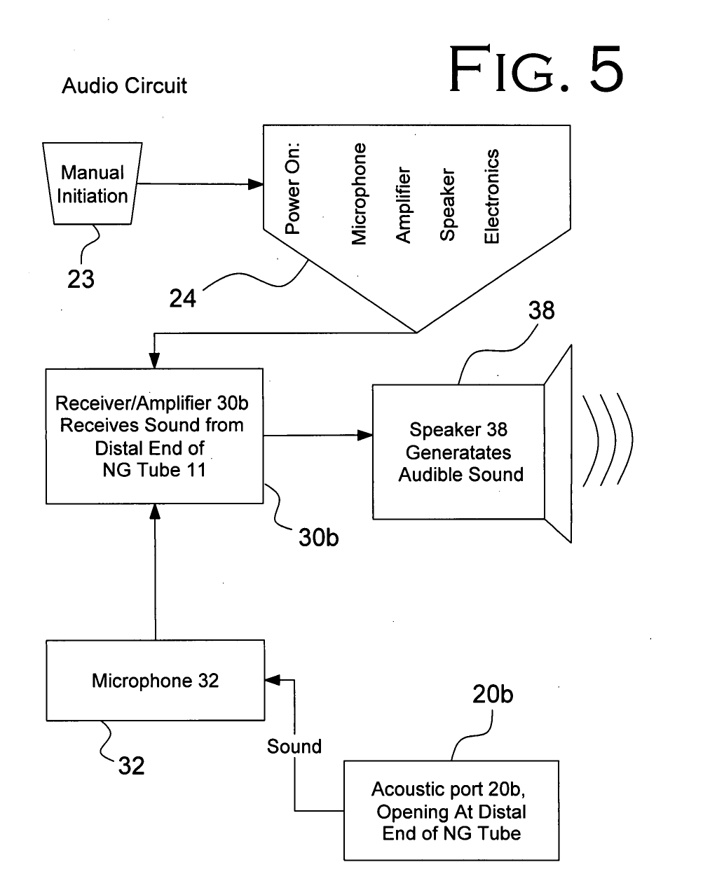

In the second embodiment, shown in FIG. 4 and FIG. 5, an audio monitoring circuit is illustrated, with the flowchart of FIG. 5 illustrating an overview of the operation of the audio monitoring circuit. The feedback initiator at the distal end of flexible tube 11 is an acoustic port 20b or opening using tube 11 as a means for sound to travel to the microphone 32 on the proximal end of tube 11, FIG. 4.

Feedback receiver 30b in the second embodiment preferably comprises a microphone 32 to receive the sounds from the distal end of flexible tube 11 configured with the appropriate amplifiers to amplify the received sounds and the appropriate circuitry to transmit the sounds via conducting means 40 to the speaker 38 which is configured with the appropriate circuitry to generate an audible sound that can be heard by the clinicians. The speaker 38 serves as a notifying device that provides the clinician with information concerning the distal end of flexible tube 11.

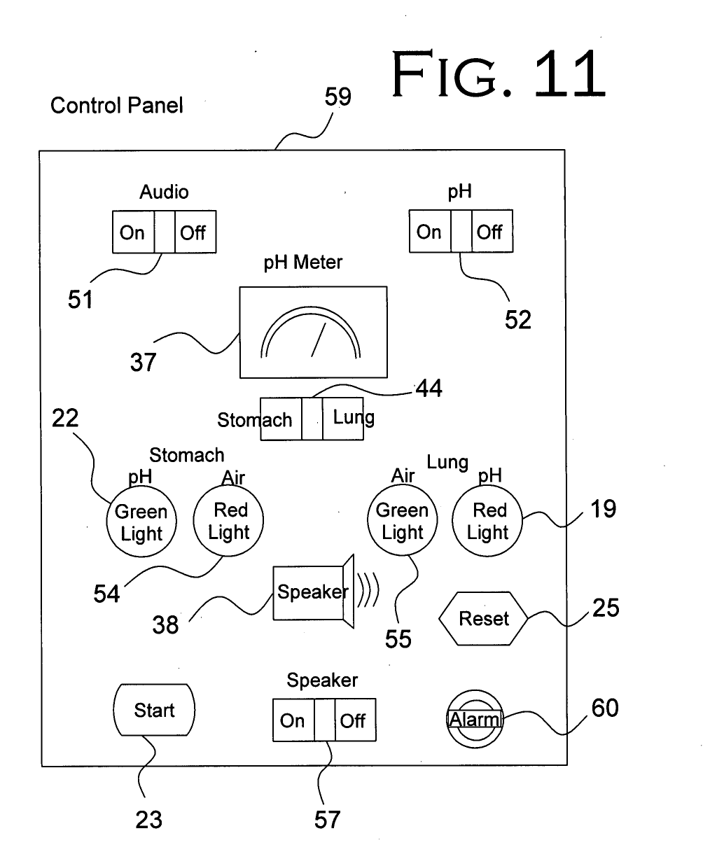

The sound reverberations and echoes from the acoustic port 20b at the distal end or flexible tube 11, detected by microphone 32, will vary depending on the size, shape, and fluid of the cavity, thereby enabling clinicians to determine the placement location merely by listening to the audible sound from the speaker 38 and differentiating between the sounds made when a tube is correctly placed in the organ of interest, and when the tube is incorrectly located. For example, if the stomach is the organ of interest, the sounds should be typical stomach sounds, such as gurgling, whereas if the tube is incorrectly placed in the lungs, trachea, or esophagus sounds of air movement may be heard. This increases staff efficiency saving time and effort, because instead of using the traditional auscultatory method requiring the staff to insufflate air through the tube and then to use a stethoscope to listen to the sounds, the present invention provides continuous audible feedback. When the audible feedback is no longer desired, speaker 38 can be manually switched off via reset button 25 (FIG. 11). The audio circuit is initiated by manual initiation button 23. Radiation exposure is reduced when placement of the nasogastric tube 11 is determined by this method instead of requiring radiological confirmation.

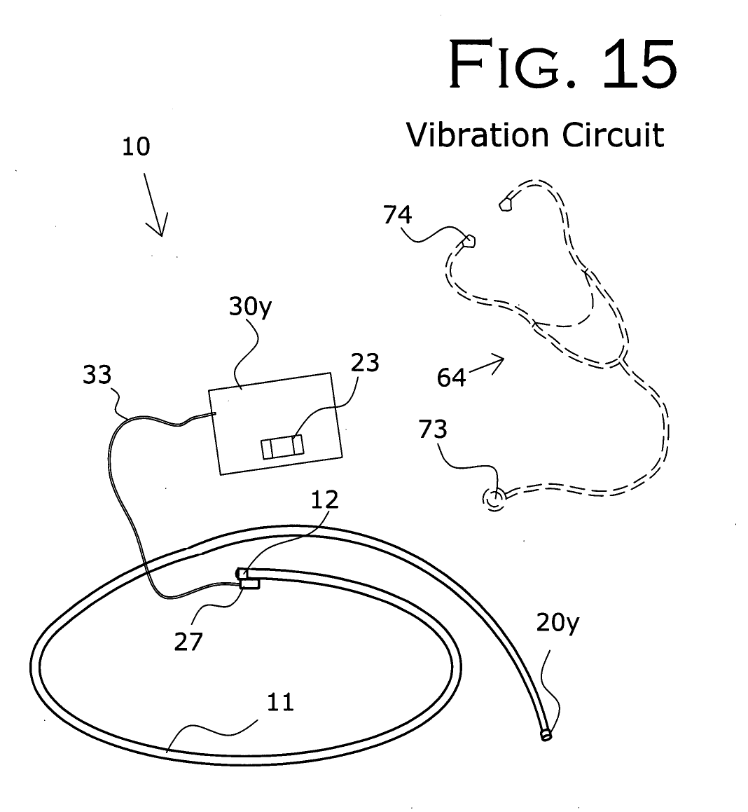

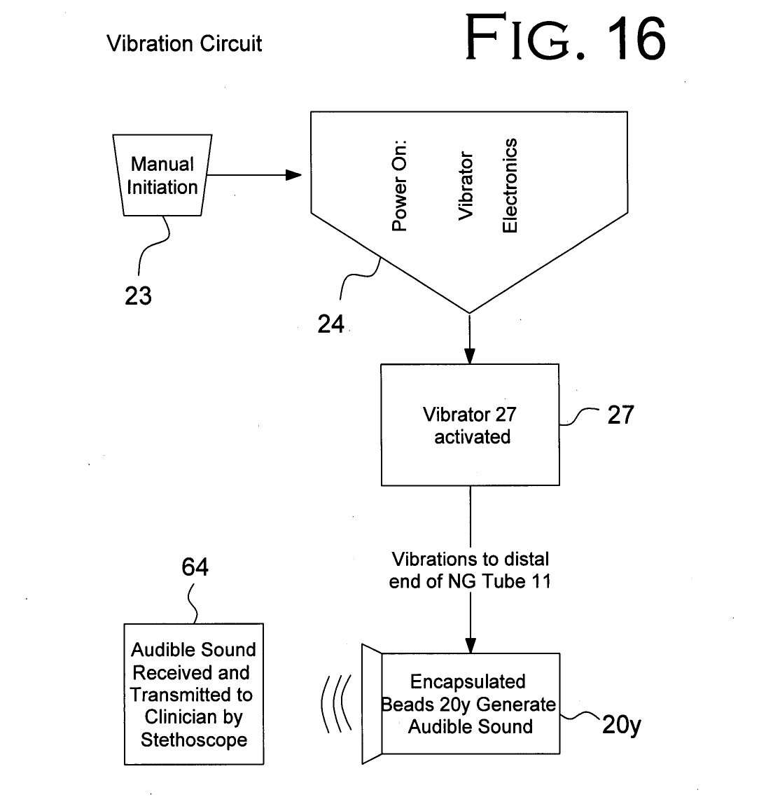

In FIGS. 15 and 16 the third embodiment, the vibration circuit, is illustrated. A vibration vibrator 27 is provided to produce a pulsation or vibration to tube 11. The feedback initiator 20 is encapsulated beads 20y, disposed at the distal end of flexible tube 11. The encapsulated beads 20y are used to assist the clinician in determining the location of the distal end of tube 11. The encapsulated beads 20y may be any small contained objects that are space-efficient and generate an audible sound by agitation or repeated concussions. The vibration and sound from encapsulated beads 20y is received by feedback receiver 30y, the diaphragm 73 of handheld stethoscope 64, and transmitted by the stethoscope tubing to the earpieces 74. The clinician receives this sound that provides information about the distal end of flexible tube 11 via the earpieces of stethoscope 64 serving as a notifying device. Alternately, the audio monitoring circuit of FIG. 4 and FIG. 5 can be utilized with this vibration circuit, in which case, microphone 32 would detect the vibration or sound and speaker 38 would serve as the notifying device to allow the clinician to hear the vibrations.

The flowchart of FIG. 16 illustrates one method of use of the vibration circuit. The vibration circuit is manually started by manual initiation button 23, with power being provided to the vibrator 27 via conducting means extended portion 33 (FIG. 15) and associated electronics as shown in block 24. In block 27, vibrator 27 sends pulses or vibrations or sounds to the distal end of flexible tube 11. The pulses are received by encapsulated beads 20y, which then generate an audible sound. The audible sound is received by a conventionally available stethoscope 64 and conveyed to the clinician.

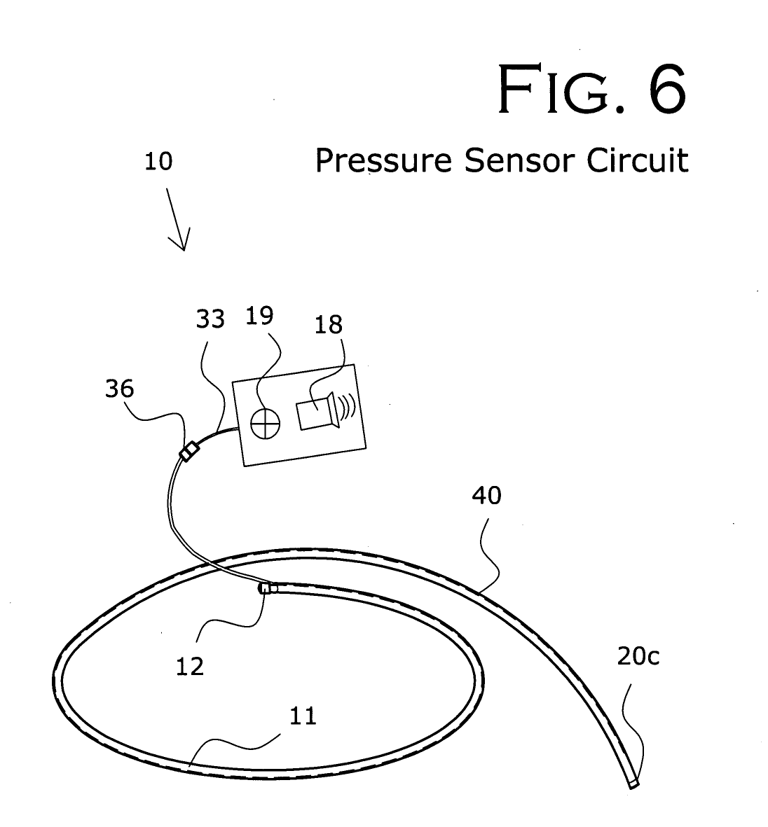

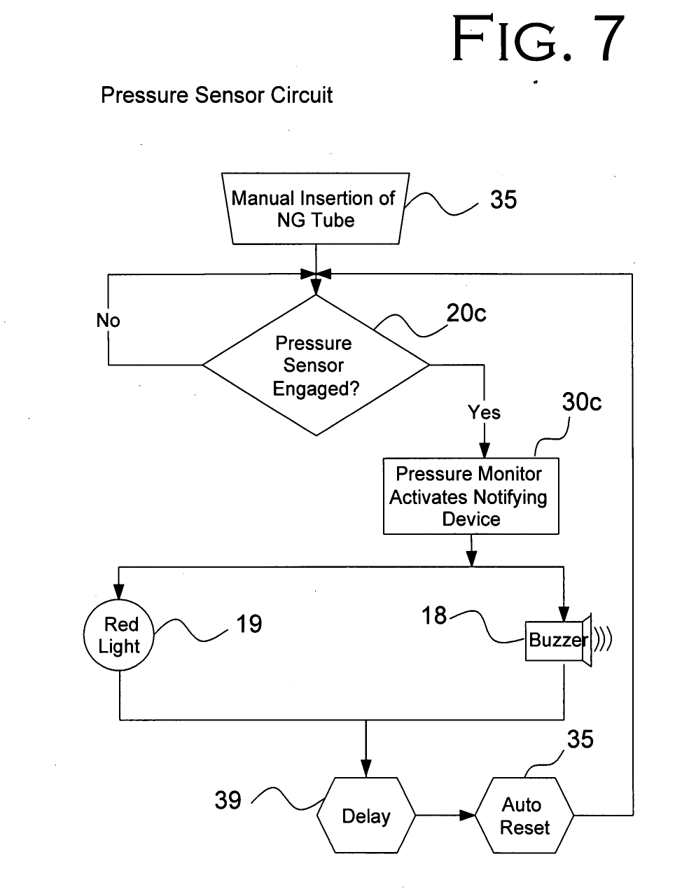

Illustrated in FIG. 6 and FIG. 7 is the fourth embodiment, a contact pressure sensor circuit to notify the attending clinician of possible injury to the patient if the contact pressure during intubation exceeds the integrity of the tissue. The feedback initiator 20c at the distal end of flexible tube 11 comprises a pressure switch 20c configured to measure the contact pressure at the distal end of flexible tube 11. Feedback receiver in this fourth embodiment comprises a pressure monitor 30c configured to receive the pressure information from pressure switch 20c and configured to activate the notifying devices, a preferably red indicator light 19 and/or warning buzzer 18, with associated electronics, if the pressure is inappropriately high. Preferably the pressure monitor 30c, the indicator light 19, and the warning buzzer 18 are incorporated into control panel 59.

Contact pressure switch 20c is configured to be activated during intubation 35 when the distal end of tube 11 is pressed or pushed against the interior of an organ of a patient with an amount of pressure that exceeds the integrity of the tissue. The activation of pressure switch 20c is transmitted to the contact pressure monitor 30c disposed at the proximal end of tube 11 via conducting means 40. If the pressure is inappropriately high, pressure monitor 30c then activates indicator light 19 and buzzer 18, either or both serving as notifying devices to notify the attending clinician. Once alerted, the clinician can modify their current approach in positioning the nasogastric tube. After a small delay 39, preferably the pressure sensor circuit is automatically reset, for the convenience of the clinician to continue the intubation procedure.

Preferably, but optionally, contact pressure switch 20c may be constructed of a generally radiopaque material, thereby allowing the contact pressure switch 20c to function as a marker for the distal end of nasogastric tube 11, thus allowing the confirmation of placement through radiology if that is deemed necessary.

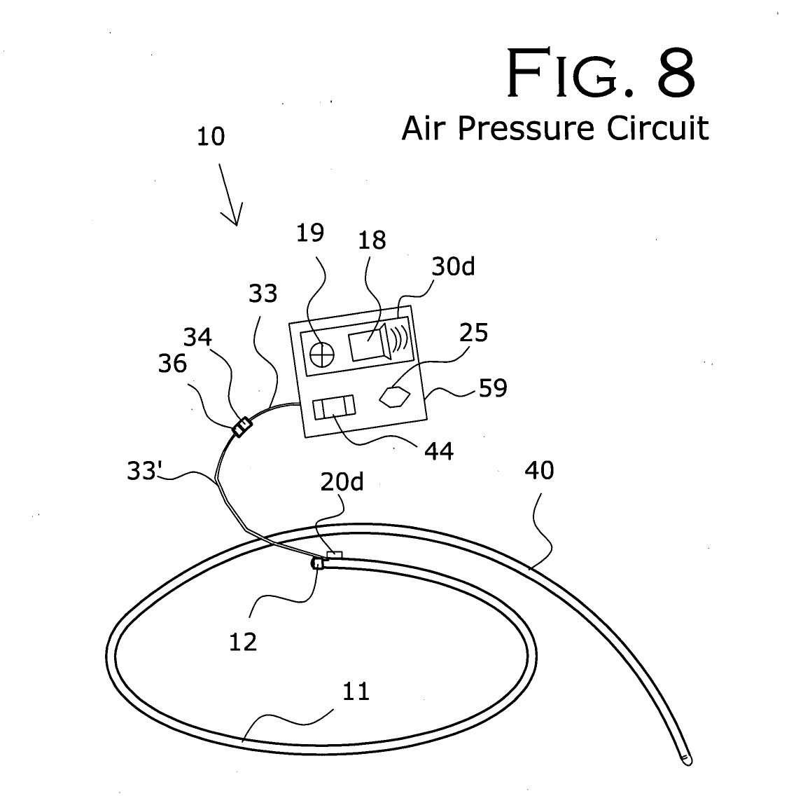

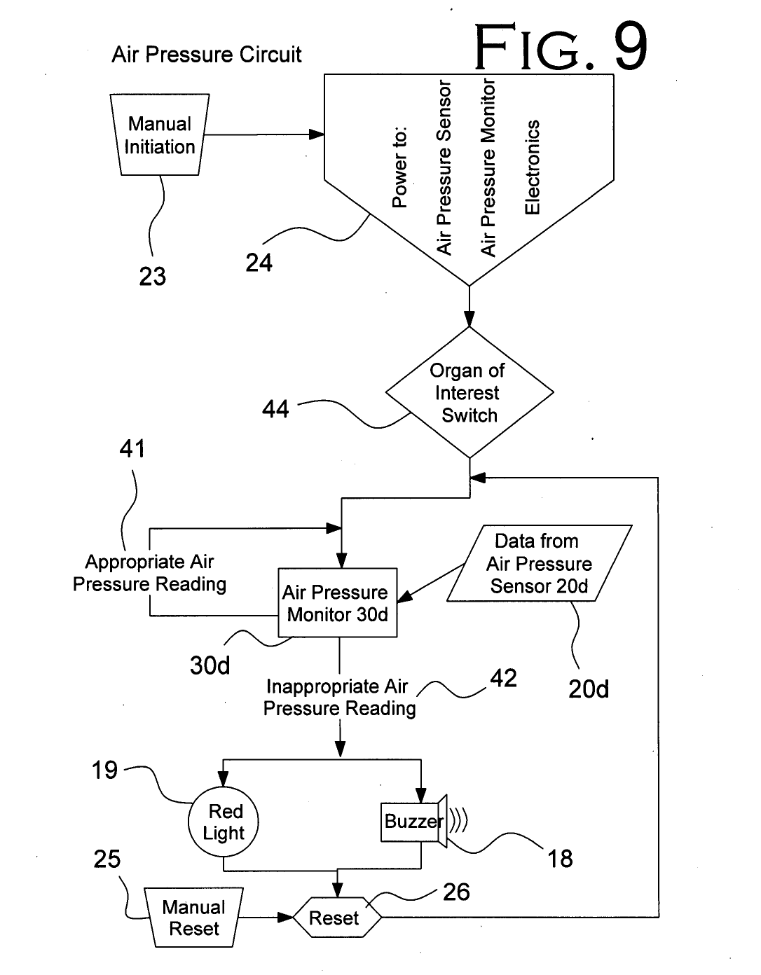

Illustrated in FIG. 8 and FIG. 9 is the fifth embodiment, an air pressure circuit to monitor air flow so that the attending clinician can ascertain whether the nasogastric tube is located in the airway or not, either during intubation or after intubation. The feedback initiator connected to the proximal end of flexible tube 11 is an air pressure sensor 20d. The air pressure sensor 20d is configured to measure the pressure or airflow of the air at the distal end of flexible tube 11. The detected air pressure data is transmitted to the feedback receiver, air pressure monitor 30d via the extended portion 33, 33′ of conducting means 40 to the feedback receiver, air pressure monitor 30d. Air pressure monitor 30d is configured with the appropriate electronics to receive the information from the air pressure sensor 20d, to analyze the information to determine if the information is appropriate for the organ of interest, and to send a signal to activate the notifying device, a preferably red indicator light 19 and warning buzzer 18, to alert the clinician of the air pressure or lack thereof at the distal end of flexible tube 11. The extended portion 33, 33′ of conducting means 40 is configured to carry the electrical signal from the air pressure sensor 20d to the air pressure monitor 30d. The extended portion 33, 33′ of conducting means 40 may directly connect the air pressure sensor 20d to the air pressure monitor 30d (not shown) or may connect through optional quick connectors 34, 36 (as shown).

Also preferably included in the fifth embodiment is an organ of interest determination switch 44 incorporated into control panel 59. The organ of interest determination switch 44 allows the attending clinician to set the circuit on either the lung or the stomach. Based on the organ of interest chosen, the circuitry and electronics are configured to display the correct reading for the chosen organ based on the data received.

A conventional air pressure sensor 20d is utilized. The air pressure sensor 20d will preferably be chosen that has a suitable air pressure range and that is space-efficient.

As illustrated in the flowchart of FIG. 9, after the manual initiation 23 of the air pressure circuit, power 24 is supplied to the various parts of the circuit, including the air pressure sensor 20d located proximally on nasogastric tube 11, feedback receiver 30d, and the other associated electronics.

Then the attending clinician manually operates the organ of interest determination switch 44 to designate the organ into which the nasogastric tube 11 will be inserted. Organ of interest determination switch 44 may optionally include a light (not shown) which will be lighted to designate the chosen organ of interest, for convenience for the clinician.

Air pressure sensor 20d then takes readings and transmits the resultant data back to the feedback receiver. The feedback receiver, an air pressure monitor 30d, is configured with the appropriate circuitry and electronics to receive the electrical output from air pressure sensor 20d and to analyze it. If the air pressure reading is appropriate 41 for the organ of interest, the air pressure sensor 20d merely continues to monitor the air pressure.

If the air pressure reading is inappropriate 42 for the organ of interest, the feedback receiver 30d causes the notifying devices to be activated. Warning buzzer 18 emits an audible alarm and/or the warning indicator light 19 is lighted, thus producing a visual and auditory signal and notification to the clinician of the problem. These may automatically reset after a given amount of time, or alternatively, as shown, they may be manually reset when the clinician depresses reset button 25, whereby the electronics reset the circuit 26.

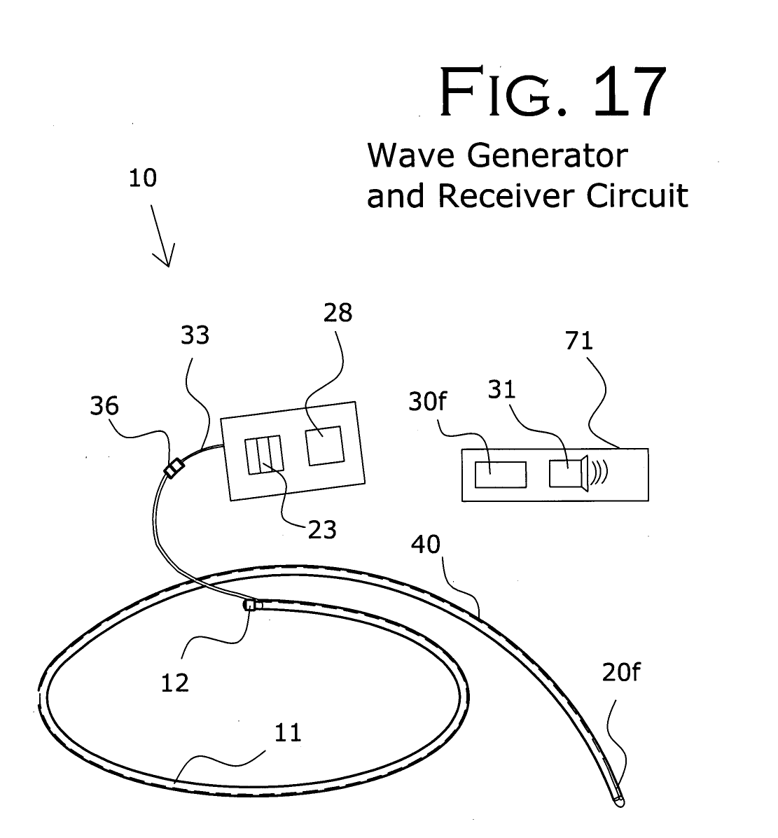

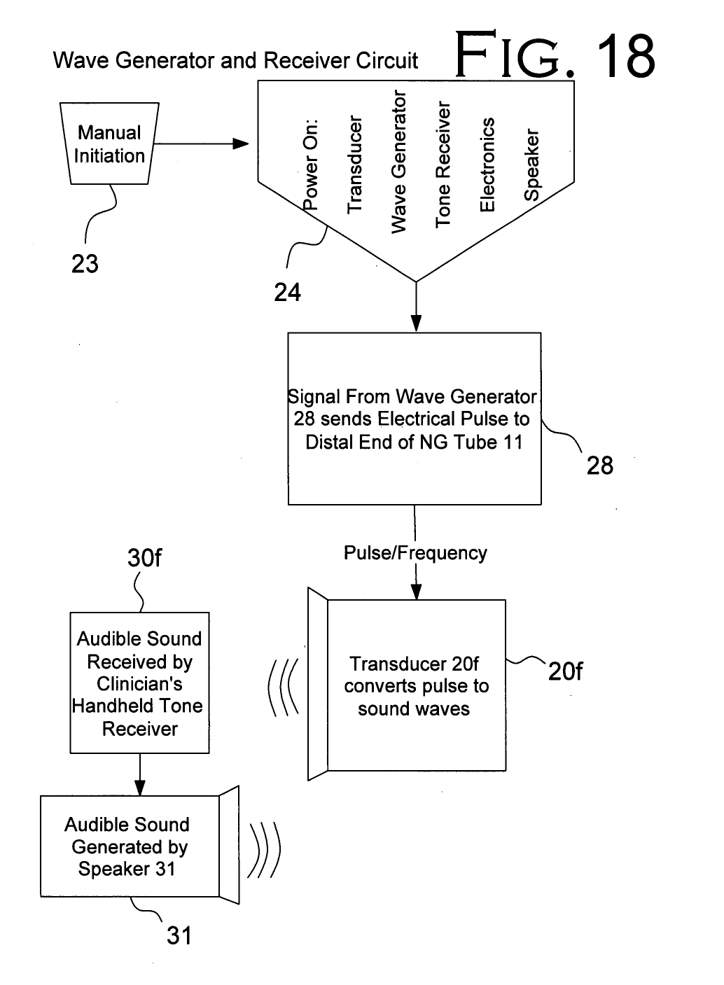

FIG. 17 and FIG. 18 illustrate a sixth embodiment of the nasogastric tube placement and monitoring system, a wave generator and receiver circuit using either mechanical waves, such as sound waves, or electromagnetic waves, such as radio waves. A wave generator 28 generates an electrical pulse which is transmitted via conducting means 40 to the feedback initiator, transducer 20f, located at the distal end of flexible tube 11. The transducer 20f converts the electrical pulse to sound waves, or to specific tones, that are then received by the feedback receiver, a tone receiver 30f. Tone receiver 30f comprises the necessary circuitry and electronics to receive the sound waves from transducer 20f and to transmit a signal representing the sound waves to the notifying device, a speaker 31. Both tone receiver 30f and speaker 31 are preferably combined into a single handheld container or handheld enclosure 71. The handheld enclosure 71 is preferably sized and shaped to be conveniently held by the attending clinician over the area of the organ of interest to optimally receive the sound waves from the transducer 20f. The sound from speaker 31 provides an indication of the location of the distal end of the flexible tube 11, not only by the volume at particular locations, but also by the echoes and vibrations incorporated into the sound from the organ of interest.

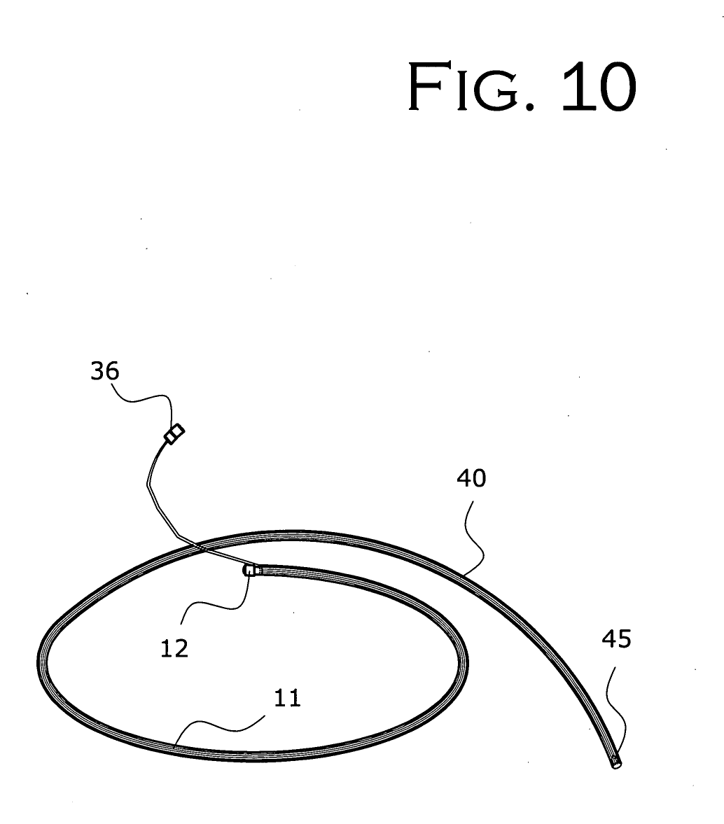

FIG. 10 illustrates a preferred type of flexible tube 11 to be used in the nasogastric tube placement and monitoring system of the present invention, providing both a crystal 45 and a variation in the material of nasogastric tube 11.

The crystal 45 is disposed at the distal end of nasogastric tube 11, either attached to tube 11 or embedded into the material of the tube 11. The crystal 45 is configured to monitor the integrity of the system's conductors, conducting means 40, which electrically connect the feedback initiator 20 to the feedback receiver 30. The crystal 45 serves as a security measure so that only standardized tubes are used with the nasogastric tube placement and monitoring system of the present invention, plus the crystal confirms the continued soundness and unimpaired operation of conducting means 40.

The compositional material of nasogastric tube 11 is preferably chosen to have a heat-sensitive color change. This material will cause the tube 11 to change color if recycling of the nasogastric tube 11 is attempted, such as by the use of an autoclave. The color change would serve as an indicator that the tube should not be used, thus preventing a negative impact on the patient's health by the use of a potentially contaminated tube 11.

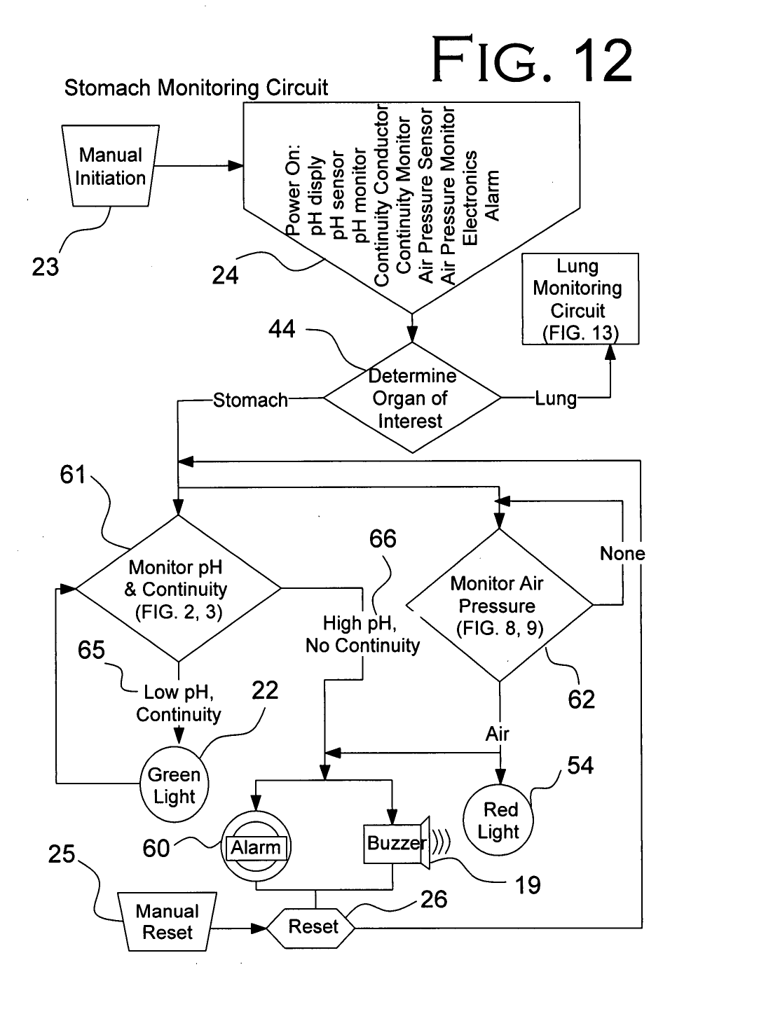

Although the first, second, third, fourth, fifth, sixth, seventh, eighth, and ninth embodiments are described separately, any combination of two or more embodiments or all of the embodiments with their associated components can be included in a single nasogastric tube and thereby used in combination, if desired. Preferably at least two of the circuits from the illustrated embodiments are included in the nasogastric tube placement and monitoring system 10 of the present invention. One such combination system, the seventh embodiment, is illustrated in FIG. 12 utilizing the control panel of FIG. 11.

FIG. 11 shows a diagrammatic illustration of a control panel 59, which can be used with any of the embodiments of the present invention, but is herein illustrated as configured for the seventh embodiment of the nasogastric tube placement and monitoring system 10. Control panel 59 is sized and configured to conveniently integrate the circuits to be utilized, to provide easily accessible controls for the circuits, and to convey the information concerning the distal end of flexible tube 11 to the attending clinician. Control panel 59 is designed to be placed near the patient's bed or other location where the intubation could be performed. The control panel elements and the electronic components of the various embodiments should preferably be digital, although analog implementations are within the scope of the present invention.

The control panel for the seventh embodiment preferably comprises the following: a start button 23 to initiate power to electronic components of the nasogastric tube placement and monitoring system; an organ of interest switch 44 to allow the clinician to manually designate the intended organ for the procedure; an audio on-off switch 51 to power the audio monitoring circuit; a pH monitoring/continuity circuit on-off switch 52 to power the pH monitoring/continuity circuit; a pH display 37 to display the sensed pH; a speaker 38 configured to output sounds; an on-off switch to power speaker 38; a manual reset button 25 to initiate the resetting of the circuits; an alarm 60 which displays a visible and preferably red light; a stomach air indicator preferably red light 54 to designate an inappropriate air pressure sensor reading; a stomach pH indicator preferably green indicator light 22 to indicate an appropriate pH reading; a lung air indicator light 55 to designate an appropriate air pressure sensor reading; and a lung pH indicator preferably red light 19 to indicate an inappropriate pH.

While several separate power and on-off switches are illustrated for demonstrative purposes, in practice a single on-off switch could incorporate the several circuits utilized, thus simplifying the initiation of the intubation system. Other notifying devices, such as other lights, buzzers, and alarms can additionally be incorporated to more easily allow the clinician to receive data about the distal end of flexible tube 11.

Similarly, while only one pH light and one air light are illustrated for the stomach or for the lung, both a red and a green light for both the pH and for the air can optionally be provided, if desired. For example, if the organ of interest switch 44 were turned to “stomach” a total of 4 lights could be made available, a green light for an appropriate pH, a red light for an inappropriate pH, a green light for an appropriate reading from the air pressure sensor, and a red light for an inappropriate reading from the air pressure sensor. If “lung” was chosen with the organ of interest switch 44, similarly, four lights could be made available, whereas only two are illustrated.

As shown in FIG. 12, the seventh embodiment of the nasogastric tube placement and monitoring system 10 of the present invention, a stomach placement and monitoring circuit, illustrates a combination of two of the previously described embodiments. This circuit would be selected by manual manipulation of the organ of interest switch 44 by choosing “stomach”. The pH monitoring/continuity circuit of FIG. 2 and FIG. 3 and the air pressure circuit of FIG. 8 and FIG. 9 are used in the stomach placement and monitoring circuit to assist the attending clinician. Optionally, the audio monitoring circuit of FIG. 4 and FIG. 5, the pressure sensor circuit of FIG. 6 and FIG. 7, the vibration circuit of FIG. 15 and FIG. 16, and/or the wave generator and receiver circuit of FIG. 17 and FIG. 18 may be additionally incorporated into this embodiment (not shown).

The operation of the stomach placement and monitoring circuit is manually initiated by engaging the start button 23, which, in block 24, powers on the air pressure sensor 20d, air pressure monitor 30d, alarm 60, pH sensor 20a, pH monitor 30a, pH display 37, continuity conductor 20e, continuity monitor 30e, and the associated electronic components. The pH monitor 30a may be separately turned on by pH switch 52 (not shown in flowchart of FIG. 12, but illustrated in FIG. 11), or, alternatively, may be included in power on (block 24), as shown in FIG. 12.

Then the organ of interest, in this case the stomach, is selected by the attending clinician by manual manipulation of the organ of interest determination switch 44.

Either or both the pH and the continuity is/are monitored in block 61 using the above described pH monitoring/continuity circuit of FIG. 2 and FIG. 3. If the pH is low and/or continuity is detected 65, green indicator light 22 is lighted to signify to the clinician that the appropriate pH and/or continuity is being sensed, and the circuit continues to monitor the pH (block 61). If the pH is high and/or no continuity is detected 66 an inappropriate pH and/or continuity is being sensed.

The air pressure is monitored (block 62) by the air pressure circuit of FIG. 8 and FIG. 9. If the air pressure signifies that the tube is in an airway passage, red warning light 54 is lighted. If the pH is high and/or no continuity is detected 66 and if the air pressure signifies that the tube 11 is in an airway passage, then alarm 60 is activated with a visible red light plus, preferably, a buzzer 19. If alarm 60 is activated, the circuits can be manually reset by depressing the reset button 25, whereby the electronics reset the circuit 26. Alternatively, if desired the circuits can automatically reset after a time delay (not shown).

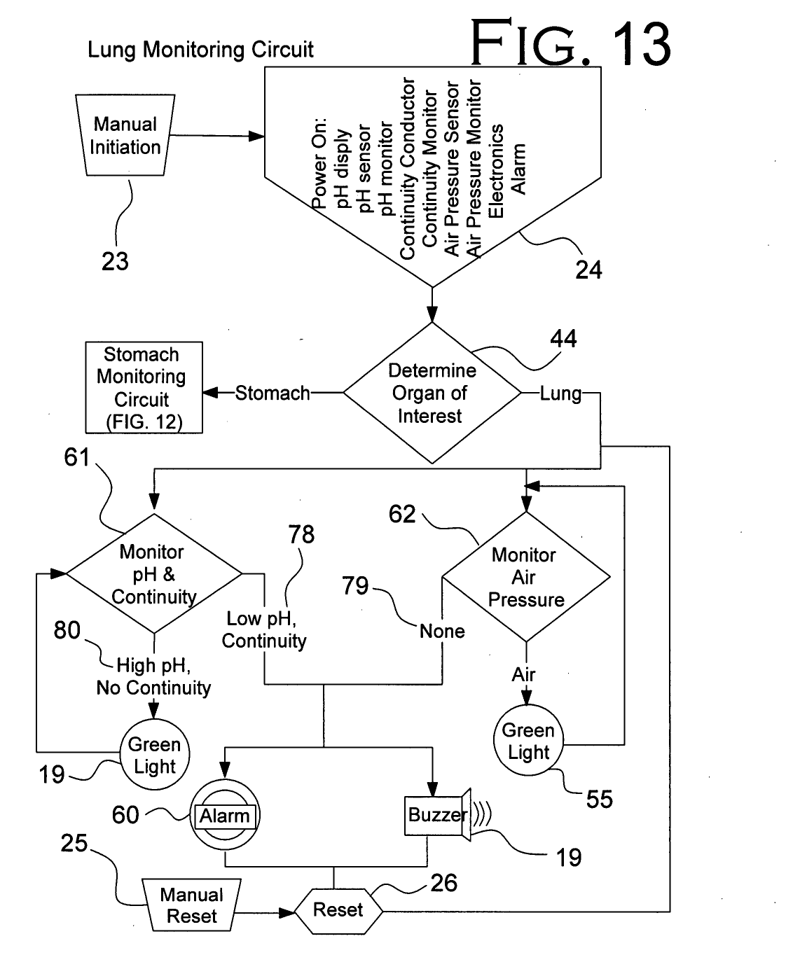

FIG. 13 illustrates the eighth embodiment, a lung placement and monitoring circuit, using the pH monitoring circuit of FIG. 2, FIG. 3 and the air pressure circuit of FIG. 8, FIG. 9. This circuit would be selected by manual manipulation of the organ of interest switch 44 by choosing “lung”.

Optionally, the audio monitoring circuit of FIG. 4 and FIG. 5, the pressure sensor circuit of FIG. 6 and FIG. 7, the vibration circuit of FIG. 15 and FIG. 16, and/or the wave generator and receiver circuit of FIG. 17 and FIG. 18 may be additionally incorporated into this embodiment (not shown).

The operation of the lung placement and monitoring circuit is manually initiated by engaging the start button 23, which powers on (block 24) the air pressure sensor 20d, air pressure monitor 30d, alarm 60, pH sensor 20a, pH monitor 30a, display 37, continuity conductor 20e, continuity monitor 30e, and associated electronic components. The pH monitor 30a may be separately turned on by pH switch 52 (not shown in flowchart of FIG. 12, but illustrated in FIG. 11), or, alternatively, may be included in power on (block 24), as shown in FIG. 12.

The organ of interest, in this case the lung, is selected by the attending clinician by manual manipulation of the organ of interest determination switch 44.

Either or both the pH and the continuity is/are monitored in block 61 using the above described pH monitoring/continuity circuit of FIG. 2 and FIG. 3. If the pH is high and/or there is no continuity 80, green indicator light 19 is lighted to signify to the clinician that the appropriate pH and/or continuity is being sensed, and the circuit continues to monitor the pH, block 61. If the pH is low and/or no continuity is detected 78, an inappropriate pH and/or continuity is being sensed.

The air pressure is monitored (block 62) by the air pressure circuit of FIG. 8 and FIG. 9. If the air pressure signifies that the tube is in an airway passage, green indicator light 55 is lighted. If the air pressure signifies that the tube is not in an airway passage and if the pH is low and/or no continuity is detected 79, alarm 60 is activated with a visible light plus, optionally a buzzer 19 may be sounded. If alarm 60 is activated, the circuits can be reset 26 by manually operating button 25. Alternatively, if desired, the circuits can automatically reset after a time delay (not shown).

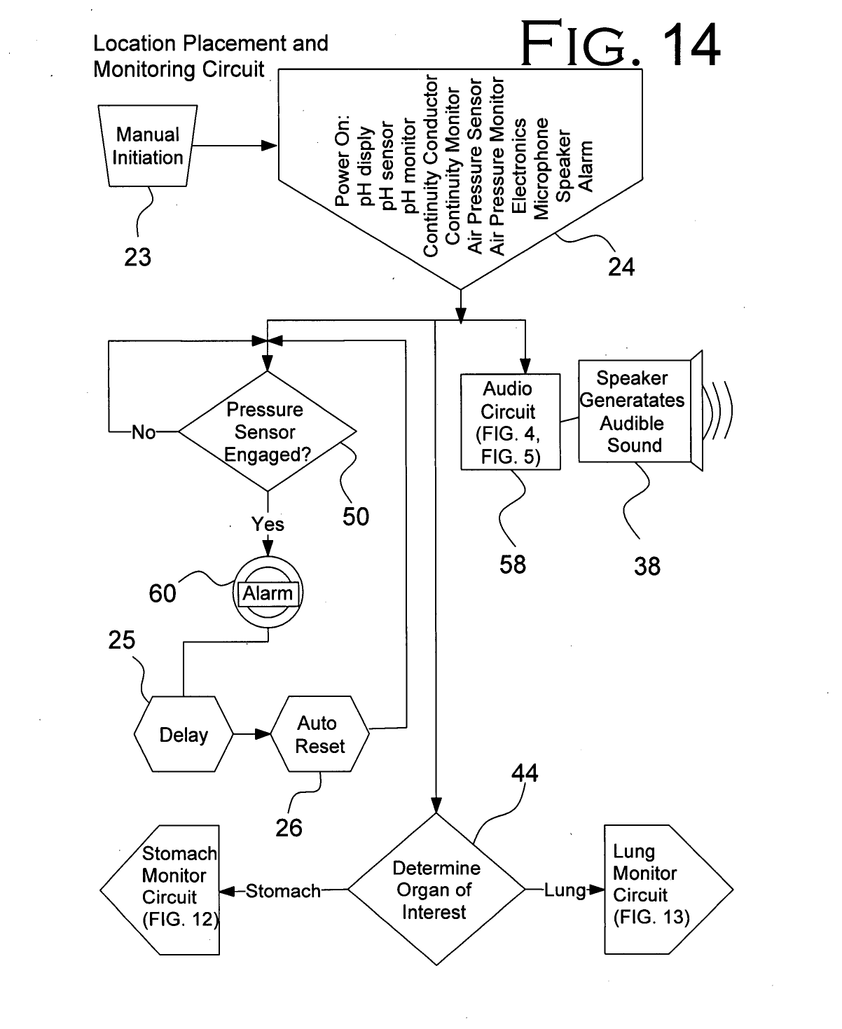

FIG. 14 illustrates combined circuits to form a nasogastric tube location placement and monitoring circuit, the ninth embodiment, utilizing the pressure circuit of FIG. 6, FIG. 7, the audio monitoring circuit of FIG. 4, FIG. 5, stomach placement and monitoring circuit of FIG. 12 (which utilizes the pH sensor/continuity circuit of FIG. 2, FIG. 3 and the air pressure circuit of FIG. 8, FIG. 9) and the lung placement and monitoring circuit of FIG. 13 (which utilizes the pH sensor/continuity circuit of FIG. 2, FIG. 3 and the air pressure circuit of FIG. 8, FIG. 9).

Manual initiation 23 powers the air pressure sensor 20d, air pressure monitor 30d, pH sensor 20a, pH monitor 30a, display 37, continuity conductor 20e, continuity monitor 30e, microphone 32, speaker 38, alarm 60, and associated electronic components. Some components can be optionally be separated powered on, such as, for example, PH switch 52 can be used to turn on the pH sensor 20a, pH monitor 30a, continuity conductor 20e, continuity monitor 30e, and pH display 37; the speaker switch 57 can turn on the speaker 38; and the audio monitoring circuit can be powered by audio switch 51.

Then the organ of interest is selected by the attending clinician by manual manipulation of the organ of interest determination switch 44.

The pressure sensor circuit 50 determines if the pressure sensor 20c is engaged, alerting the clinician to a problem via alarm 60, as described in FIG. 6, FIG. 7.

The audio circuit 58 generates audible sounds via speaker 38, as described in FIG. 4, FIG. 5.

After the determination of the organ of interest 44, either the stomach placement and monitoring circuit of FIG. 12 or the lung placement and monitoring circuit of FIG. 13 is activated. Both the stomach placement and monitoring circuit and the lung placement and monitoring circuit utilize the air pressure circuit and the pH monitoring/continuity circuit.

The pH and/or continuity is monitored and displayed using the pH monitoring/continuity circuit, with the clinician alerted to an appropriate pH and continuity or to an inappropriate pH and continuity, as described in FIG. 6 and FIG. 7.

The air pressure is monitored via the air pressure sensor 20d using the air pressure circuit as described in FIG. 8 and FIG. 9, with the clinician alerted to an appropriate amount of sensed air or to an inappropriate amount of sensed air.

As with the above embodiments, if alarm 60 is activated, the circuits can be manually reset by depressing the reset button 25, whereby the electronics reset the circuit 26. Alternatively, if desired the circuits can automatically reset after a time delay (not shown).

Optionally, on any of the embodiments of the present invention, one way valves can be provided within the tube 11, thereby assisting in removing fluids or materials from the lungs or stomach.

While a limited number of combinations of the provided circuits have been illustrated, additionally, many other combinations of two or more of the provided circuits can be utilized to provide information to the clinician about the distal end of the tube 11.

Thus by the utilization of one or a combination of the provided circuits, the information about the location of the distal end of the tube 11 is monitored during intubation and as long as the tube is inserted. If the distal end moves out of the organ of interest, alarm 60 lights up and, preferably, emits an audible warning sound, so corrective measures can be taken by the attending staff member. The provided nasogastric tube placement and monitoring system eliminates the need to use the “blind” placement techniques currently in use and thus minimizes staff errors.

From the foregoing, it will be apparent that the nasogastric tube placement and monitoring system 10 of the current invention provides an efficient system that assists both in the intubation and in the long-term monitoring of the tube, which can be utilized either in the stomach and the lungs. The nasogastric tube placement and monitoring system 10 of the present invention improves staff efficiency while reducing the patient's exposure to radiation as well as reducing the cost, by providing a system that confirms the placement location of the nasogastric tube without radiological confirmation.

In addition, the present invention, although described for use as a nasogastric tube with the desired placement location being the patient's stomach or lung, can be used for other similar intubation procedures, for example where the tube is introduced through the mouth or where the desired placement of the tube is not in the stomach or lung, but instead might be in the small intestine.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

I claim:

1. A nasogastric tube placement and monitoring system for use by a clinician during intubation of a patient, comprising:
a flexible tube having a distal end, a proximal end, and an extended midsection, said flexible tube being adapted and sized for insertion into a nasal passage of said patient;
at least two feedback initiators configured to provide information concerning the location of said distal end of said flexible tube;
at least two feedback receivers configured to receive said information from said at least two feedback initiators;
a conducting means connecting one of said at least two feedback initiators to one of said at least two feedback receivers and configured to transport said information from one of said at least two feedback initiators to one of said at least two feedback receivers; and
at least one notifying device configured to present said information concerning the location of said distal end of said flexible tube to said clinician.

2. The nasogastric tube placement and monitoring system for use by a clinician during intubation of a patient, as recited in claim 1, wherein one of said at least two feedback initiators comprises a pH sensor, wherein at least one of said at least two feedback receivers comprises a pH monitor, and wherein said at least one notifying device comprises a pH display.

3. The nasogastric tube placement and monitoring system for use by a clinician during intubation of a patient, as recited in claim 2, wherein said at least one notifying device further comprises an indicator light.

4. The nasogastric tube placement and monitoring system for use by a clinician during intubation of a patient, as recited in claim 1, wherein one of said at least two feedback initiators comprises a continuity conductor and wherein at least one of said at least two feedback receivers comprises a continuity monitor.

5. The nasogastric tube placement and monitoring system for use by a clinician during intubation of a patient, as recited in claim 1, wherein one of said at least two feedback initiators comprises an acoustic port configured to transport sound from said distal end of said flexible tube to said proximal end of said flexible tube, wherein at least one of said at least two feedback receivers comprises a microphone, and wherein said at least one notifying device comprises a speaker.

6. The nasogastric tube placement and monitoring system for use by a clinician during intubation of a patient, as recited in claim 1, wherein one of said at least two feedback initiators comprises a contact pressure switch, wherein at least one of said at least two feedback receivers comprises a contact pressure monitor, and wherein said at least one notifying device comprises a buzzer configured to generate an audible sound.

7. The nasogastric tube placement and monitoring system for use by a clinician during intubation of a patient, as recited in claim 6, wherein said at least one notifying device further comprises a warning light.

8. A nasogastric tube as recited in claim 6, wherein said contact pressure switch is constructed of a generally radiopaque material.

9. The nasogastric tube placement and monitoring system for use by a clinician during intubation of a patient, as recited in claim 1, wherein one of said at least two feedback initiators comprises encapsulated beads disposed at said distal end of said flexible tube, wherein at least one of said at least two feedback receivers comprises a stethoscope.

10. The nasogastric tube placement and monitoring system for use by a clinician during intubation of a patient, as recited in claim 1, wherein one of said at least two feedback initiators comprises a transducer, wherein at least one of said at least two feedback receivers comprises a tone receiver, and wherein said at least one notifying device comprises a speaker.

11. The nasogastric tube placement and monitoring system for use by a clinician during intubation of a patient, as recited in claim 1, wherein one of said at least two feedback initiators comprises an air pressure sensor, wherein at least one of said at least two feedback receivers comprises an air pressure monitor, and wherein said at least one notifying device comprises an indicator light.

12. The nasogastric tube placement and monitoring system for use by a clinician during intubation of a patient, as recited in claim 1, wherein said flexible tube is formed of a heat indicating material.

13. The nasogastric tube placement and monitoring system for use by a clinician during intubation of a patient, as recited in claim 12, further comprising a quick connector.

14. A nasogastric tube for use in intubation of a patient, comprising:
a flexible tube having a distal end, a proximal end, and an extended midsection, said flexible tube being adapted and sized for insertion into a nasal passage of said patient;
a conducting means disposed generally along the length of said flexible tube and extending past the proximal end of said flexible tube, said conducting means configured to conduct electrical signals from said distal end of said flexible tube to said proximal end of said flexible tube; and
a pressure switch disposed at said distal end of said flexible tube and configured to measure the pressure at said distal end of said flexible tube.

15. The nasogastric tube for use in intubation of a patient, as recited in claim 14 wherein said pressure switch is formed of a generally radiopaque material.

16. The nasogastric tube for use in intubation of a patient, as recited in claim 15 wherein said flexible tube is formed of a heat indicating material, wherein said heat indicating material visually indicates when said flexible tube has been exposure to high heat.

17. The nasogastric tube for use in intubation of a patient, as recited in claim 14 further comprising multiple encapsulated beads disposed at said distal end of said flexible tube and configured to produce a sound.

18. The nasogastric tube for use in intubation of a patient, as recited in claim 17 further comprising a crystal configured to monitor conductivity at said distal end of said flexible tube.

19. The nasogastric tube for use in intubation of a patient, as recited in claim 18 further comprising electrical shielding for said conducting means.

20. The nasogastric tube for use in intubation of a patient, as recited in claim 19, further comprising an air pressure sensor disposed on said proximal end of said flexible tube.

21. The nasogastric tube for use in intubation of a patient, as recited in claim 20, further comprising a pH sensor disposed on said distal end of said flexible tube, said pH sensor configured to produce a voltage directly related to the hydrogen ion concentration of the fluid at the distal end of said flexible tube.

22. The nasogastric tube for use in intubation of a patient, as recited in claim 21, further comprising a continuity circuit configured to test the integrity of said continuity circuit.

23. The nasogastric tube for use in intubation of a patient, as recited in claim 22, further comprising a microphone disposed on the proximal end of said flexible tube 11.

24. The nasogastric tube for use in intubation of a patient, as recited in claim 23, further comprising a transducer disposed on the distal end of said flexible tube 11, said transducer configured to convert electrical pulses to sound waves.

25. The nasogastric tube for use in intubation of a patient, as recited in claim 24, wherein said conducting means comprises a quick connector disposed within the portion of said conducting means that extends past the proximal end of said flexible tube, said quick connector configured to provide electrical connectivity when connected and configured to easily disconnect.

26. A nasogastric tube placement and monitoring system for use by a clinician during intubation of a patient, comprising:
a flexible tube having a distal end, a proximal end, and an extended midsection, said flexible tube being adapted and sized for insertion into a nasal passage of said patient;
a control panel comprising an enclosure for electronics, said control panel comprising a pH display and a warning buzzer;
a conducting means disposed generally along the length of said flexible tube and having an extended portion extending beyond said proximal end of said flexible tube to said control panel, said conducting means configured to electrically conduct information about said distal end of said flexible tube from said distal end of said flexible tube to said control panel;
a pressure switch disposed at said distal end of said flexible tube and capable of detecting an excessive contact pressure at said distal end of said flexible tube, said pressure switch electrically connected to said conducting means, wherein said conducting means conducts said detected excessive contact to said control panel, and wherein said warning buzzer is configured to notify said clinician of said detected excessive contact pressure; and
a pH sensor disposed at said distal end of said flexible tube and electrically connected to said conducting means, said pH sensor capable of detecting the pH of the fluid at said distal end of said flexible tube and capable of outputting a detected pH signal, wherein said conducting means conducts said detected pH to said control panel, and wherein said pH display disposed on said control panel is configured to receive said detected pH signal and to display said detected pH signal.

27. The nasogastric tube placement and monitoring system for use by a clinician during intubation of a patient, as recited in claim 26, further comprising:
multiple pH indicator lights disposed on said control panel;
an organ of interest determination switch disposed in said control panel and configured to allow said clinician to manually select an organ of interest, said organ of interest determination switch configured to output an organ of interest signal indicating the organ of interest selected by said clinician; and
a pH monitor configured to receive said detected pH signal, configured to receive said organ of interest signal, and configured to analyze said detected pH signal based on said organ of interest signal to produce a pH analysis, wherein said pH analysis is output as a notification to said clinician via one of said multiple pH indicator lights.

28. The nasogastric tube placement and monitoring system for use by a clinician during intubation of a patient, as recited in claim 27, further comprising:
an air pressure sensor disposed at said proximal end of said flexible tube and connected to said extended portion of said conducting means, said air pressure sensor configured to detect the air pressure in said flexible tube and to output a detected air pressure signal; and
an air pressure monitor disposed in said control panel configured to receive said detected air pressure signal from said air pressure sensor via said extended portion of said conducting means, configured to receive said organ of interest signal, configured to analyze said detected air pressure signal based on said organ of interest signal, and configured to output an air pressure notification to said clinician of said detected air pressure via said warning buzzer.

29. The nasogastric tube placement and monitoring system for use by a clinician during intubation of a patient, as recited in claim 28, further comprising multiple air pressure indicator lights disposed on said control panel, wherein air pressure monitor outputs said air pressure notification to said clinician of said detected air pressure via one of said multiple air pressure indicator lights.

30. The nasogastric tube placement and monitoring system for use by a clinician during intubation of a patient, as recited in claim 29, further comprising:
an acoustic port configured to transport sound from said distal end of said flexible tube to said proximal end of said flexible tube;
a microphone configured to receive said sound from said acoustic port, said microphone disposed on said distal end of said flexible tube; and
a speaker configured to receive said sound from said microphone and to project said sound to said clinician in an audible manner.

31. The nasogastric tube placement and monitoring system for use by a clinician during intubation of a patient, as recited in claim 30, further comprising:
a continuity conductor disposed on said distal end of said flexible tube, said continuity conductor electrically connected to said conducting means, said continuity conductor configured to detect the continuity of the circuit; and
a continuity monitor disposed in said control panel and configured to receive data from said continuity conductor.

32. The nasogastric tube placement and monitoring system for use by a clinician during intubation of a patient, as recited in claim 31, further comprising:
a wave generator disposed in said control panel and configured to generate an electrical signal;
a transducer disposed on said distal end of said flexible tube and configured to receive said electrical signal and to convert said electrical signal to sound waves;
a handheld enclosure capable of being held in said clinicians hand;
a tone receiver to receive said sound waves disposed within said handheld enclosure; and
a handheld enclosure speaker disposed within said handheld enclosure and configured to receive said sound waves from said tone receiver and to project said sound waves to said clinician in an audible manner.

33. The nasogastric tube placement and monitoring system for use by a clinician during intubation of a patient, as recited in claim 32, further comprising:
a vibrator disposed on said proximal end of said flexible tube, said vibrator configured to produce a pulsation in said flexible tube; and
encapsulated beads disposed at said distal end of said flexible tube and configured to vibrate due to said pulsation from said flexible tube.

34. The nasogastric tube placement and monitoring system for use by a clinician during intubation of a patient, as recited in claim 33, wherein the pressure sensor comprises generally radiopaque material.

35. The nasogastric tube placement and monitoring system for use by a clinician during intubation of a patient, as recited in claim 34 further comprising a crystal attached to said distal end of said flexible tube configured to monitor the integrity of said conducting means and of said continuity conductor.

36. The nasogastric tube placement and monitoring system for use by a clinician during intubation of a patient, as recited in claim 35, wherein said flexible tube is formed of a heat indicating material.

37. The nasogastric tube placement and monitoring system for use by a clinician during intubation of a patient, as recited in claim 36, further comprising
electrical shielding for said conducting means; and
a reset button disposed in said control panel and configured for manual manipulation by said clinician to reset said electronics in said control panel.
